(19) United States
(12) Patent Application Publication
(10) Pub. No.: US 2022/0370050 A1
(43) Pub. Date: Nov. 24, 2022

(54) MEDICAL NEEDLE GUIDANCE SYSTEM

(71) Applicant: Eric Ross Litman, West Bloomfield, MI (US)

(72) Inventors: Eric Ross Litman, West Bloomfield, MI (US); Kyle Austin Fouty, Grand Rapids, MI (US)

(73) Assignee: Eric Ross Litman, West Bloomfield, MI (US)

(21) Appl. No.: 17/751,514

(22) Filed: May 23, 2022

Related U.S. Application Data
(60) Provisional application No. 63/191,542, filed on May 21, 2021.

(51) Int. Cl. A61B 10/02; A61B 8/08

(52) U.S. Cl. CPC A61B 10/0233; A61B 8/0841; A61B 2010/0225

(57) ABSTRACT

An insertion tool guide for a medical procedure like a biopsy. The tool guide includes (a) a frame configured to be adjoined to a medical image modality and maintain an insertion angle aligned with a plane of view generated by the medical image modality; (b) a guide body having an adjustable channel configured to receive and translate an insertion tool; (c) a main channel defined by the frame configured to allow the guide body and the insertion tool to rotate within a plane of movement; (d) a guide channel formed along one side of the frame configured to allow the guide body to translate along its length; and (e) a peg extending from the guide body and through the guide channel to secure the guide body within the frame. The guide body is configured to rotate about an axis defined by the peg.

### CROSS REFERENCE TO RELATED APPLICATION

This application claims priority to U.S. Provisional Application No. 63/191,542 filed May 21, 2021 and titled Medical Needle Guidance System. The present application claims priority to this application which are hereby incorporated herein by reference in their entirety.

### FIELD

The present disclosure relates generally to a medical device for use with syringes, probes, cannulas, and the like.

### BACKGROUND

The statements in this section merely provide background information related to the present disclosure and may not constitute prior art.

Needle guide systems for use in medical applications are known. Published application WO2020/181388 to Wen et al. titled “Needle Guide for an Angled Endocavity Transducer” provides for a needle guide for an angled ultrasound probe having a port tower with a plurality of needle ports aligned with an angled ultrasonic imaging plane. The needle ports in a port tower are aligned at the offset angle of the angled ultrasound probe and can support and guide a needle at the offset angle. Adjustment of the port tower along the transducer axis of the angled ultrasound probe maintains the alignment of the needle ports relative to the ultrasonic imaging plane and provides adjustment to accommodate differently sized patients. A method is also provided to securely align a needle with an angled ultrasonic imaging plane with an angled ultrasound probe such that the needle can accurately transect the ultrasound imaging plane.

Published patent application US2007/0233157 to Mark et al., titled “Flexible Needle Guide”, provides for a flexible needle guide having a support member and a body portion defined by a distal end and a proximal end. The proximal end is connected to the support member. The body portion further includes at least one receiving channel extending therethrough. The receiving channel includes an axis extending therethrough. The body portion is at least partially constructed from a flexible material such that instruments inserted through the receiving channel may be selectively oriented at an angle with respect to the axis extending through the receiving channel.

Published patent application US2011/0257594 to Lacoursiere et al., titled “Needle Guide”, provides for a needle guide for guiding the insertion of a needle in a patient. The needle guide includes a base element having an abutment section for abutting against the patient and a spacer extending from the abutment section. It further includes a guiding element defining at least one guiding aperture extending therethrough for inserting the needle therethrough. The guiding element is removably attachable to the spacer and movable between a distal position and a proximal position. In the distal position, the guiding element is attached to the spacer in a spaced apart relationship relatively to the abutment section, and, in the proximal position, the guiding element is detached from the spacer and substantially adjacent to the base element.

U.S. Pat. No. 10,702,303 to Andrew Neice, titled “Retreating Stop Ultrasound Needle Guide”, is directed to retreating stop ultrasound needle guide systems. The needle guide system includes a guiding component which can be positioned at different locations and angles on a mounting component. The mounting component can be coupled to a distal end of an ultrasound probe. When positioned at the different locations and angles on the mounting component, the guiding component can guide a needle through the skin of a patient at a constant point such that when the needle is advanced so that a hub of the needle reaches a stop of the guiding component, a tip of the needle is positioned directly underneath the ultrasound at a pre-selected depth.

A need still remains for effective solutions to maintain consistent insertion at desired angles.

### SUMMARY

The present disclosure provides for an insertion tool guide for a medical procedure. The tool guide includes (a) a frame configured to be adjoined to a medical image modality and maintain an insertion angle aligned with a plane of view generated by the medical image modality; (b) a guide body defining an entry port and an adjustable channel configured to receive and translate an insertion tool; (c) a main channel defined by the frame configured to allow the guide body and the insertion tool to rotate within a plane of movement; (d) a guide channel formed along one side of the frame configured to allow the guide body to translate along its length; and (e) a peg extending from the guide body and through the guide channel to secure the guide body within the frame. The guide body is configured to rotate about an axis defined by the peg. The insertion tool can be selected from the group consisting of a needle, a biopsy needle, a cannula, a catheter, and combinations thereof. The medical image modality can be selected from the group consisting of an ultrasound having an ultrasound probe, a computerized tomography (CT) scan, magnetic resonance imaging (MRI), a positron emission tomography (PET) scan, and combinations thereof.

In an example, the medical image modality is an ultrasound having an ultrasound probe and the tool guide further includes one or more attachment arms extending from the frame to contact and guide the ultrasound probe to its intended position in relation to the frame. The attachment arm can be configured to align the frame to maintain the insertion tool within the plane of view generated by the ultrasound probe. The attachment arm can also be configured to withstand a preset amount of force applied to the probe to maintain the frame in contact with or adjoined to the probe and disengage from the frame when the force surpasses the preset amount preventing unintended movement of the insertion tool during use. In an example, the attachment arm includes a connector peg and defines a protruding V-feature for matching a mating indent V-feature defined on the frame.

In yet another example, an insertion tool guide further includes a lock extending from the peg configured to prevent translational movement of the insertion tool as it passes through the adjustable channel and configured to be activated and restrict or prevent movement of the insertion tool. The guide body is configured to rotate about an axis defined by the peg. A removable guide body can be provided wherein a that is interchangeable with a different guide body defining a different diameter of the entry port to accommodate a corresponding gauge of an insertion tool. The peg is configured to secure the guide body to the frame such that the guide body is configured to slidably travel through the guide channel. The peg can be removably attached to the guide body and the guide body can be removably attached to the frame.

In still another example, the insertion tool guide further includes a plurality of feet positioned on a lower side of the frame formed of a material configured to adhere to the surface of a target area. The feet are optionally removable.

The insertion tool can be a biopsy needle configured for use with a biopsy procedure. The biopsy procedure is configured to obtain a biopsy sample of a target tissue selected from the group consisting of a liver, a breast, lung, kidney, thyroid, and combinations thereof.

The present disclosure provides for a medical biopsy process using the insertion tool guide as described herein. The process includes the steps of: (a) providing the insertion tool guide and the biopsy needle; (b) making a small incision on a tissue surface to allow for biopsy needle to access a target region; (c) inserting the biopsy needle using the insertion tool guide and a corresponding medical image modality; and (d) obtaining a sample of the target region. The process can be repeated by reinserting the biopsy needle to obtain a plurality of samples from different locations of the target region. The biopsy procedure can be configured to obtain a biopsy sample of a target tissue selected from the group consisting of a liver, a breast, lung, kidney, thyroid, and combinations thereof. In an example, the biopsy procedure is a coaxial biopsy including a step of inserting and placing a guide needle prior to threading a biopsy needle through the guide needle in order to obtain the sample.

The present disclosure further provides for an insertion tool guide system for a medical procedure including: (a) a frame configured to be adjoined to a medical image modality and maintain an insertion angle aligned with a plane of view generated by the medical image modality; (b) a guide body defining an entry port and an adjustable channel configured to receive and translate a biopsy needle in a first direction; (c) a main channel defined by the frame configured to allow the biopsy needle to rotate within a plane of movement; (d) a guide channel formed along one side of the frame configured to translate the guide body along its length; (e) a peg extending through the guide channel and attached to the guide body; wherein, the guide body is configured to rotate about an axis defined by the peg; (f) a lock configured to prevent translational movement of the biopsy needle as it progresses through the adjustable channel and to be activated and restrict or prevent movement of the insertion tool.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings in which:

FIG. 1 is a schematic illustration showing a needle guide apparatus according to the present disclosure in use with a biopsy tool, the apparatus having a single port and an adjustable channel in a first position.

FIG. 2 shows the needle guide apparatus of FIG. 1 with the channel in a different position.

FIG. 3A is an entry side schematic view of the apparatus of FIG. 1 having an entry port.

FIG. 3B is an example exit side schematic view of an apparatus of the present disclosure having an adjustable exit channel.

FIG. 3C is an example exit side schematic view of an apparatus of the present disclosure having a plurality of exit ports.

FIG. 3D is an example side view of an apparatus with a plurality of fixed channels extending from an entry port to different exit ports.

FIG. 4A is a schematic illustration showing an example apparatus according to the present disclosure connected to an ultrasound probe and having a plurality of entry ports.

FIG. 4B is an entry side schematic view of the apparatus of FIG. 4A having a plurality of entry ports.

FIG. 5 is a magnified view of an adjustment tool of the apparatus of the present disclosure.

FIG. 6 is a perspective view of an example apparatus according to the present disclosure having a radial movement channel.

FIG. 7 is a cutaway view of the apparatus of FIG. 6.

FIG. 8 is a perspective view of the apparatus of FIG. 6 illustrating the ultrasonic plane of view of a complementary ultrasonic probe.

FIG. 9 is a side view of the apparatus of FIG. 6 illustrating the radial movement channel and securing peg.

FIG. 10 is an underside view of the apparatus of FIG. 6.

FIG. 11 is an isolated view of a guide body of the apparatus of FIG. 6.

FIG. 12 is a cut-away view of the guide body from FIG. 11 showing an adjustment channel.

FIG. 13 is an example of the apparatus of FIG. 6 positioned against a probe having an attachment arm and having a V-feature indent for receiving a second attachment arm.

FIG. 14 is a perspective view of the attachment arm of FIG. 13 showing a matching V-feature protrusion.

FIG. 15 is the apparatus of FIG. 13 having two attachment arms.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The present disclosure provides for an apparatus, system, and method configured to cooperate and work with existing medical instruments (e.g. an ultrasound probe or a biopsy needle) to guide an insertion tool like a needle, probe or cannula, to a target location during a medical procedure. This can be effective for various medical procedures including but not limited to: cannulation of the internal jugular vein (IJV) or subclavian vein (SCV), or an ultrasound-guided biopsy or coaxial biopsy of the breast, liver, lung, thyroid, or kidney. The apparatus may also be attached to a patient (e.g. skin clamp, skin tape) during the medical procedure.

Often during certain invasive medical procedures like a tissue biopsy, a medical professional (i.e., doctor, nurse, specialist, etc.) is performing the procedure “free hand” without any structural guidance to ensure proper angle of insertion, insertion depth, and execution—like obtaining a tissue biopsy or sample or delivering a medication. Even with the help of visual aids like radiology tools, human error is a significant factor and mistakes are often made. For example, the 20% risk of pneumothorax aka “dropping the lung” currently associated with transthoracic lung biopsies. Of those patients that develop a pneumothorax, 5-18% require chest tube placement. The apparatus, system and method of the present disclosure aims to solve these shortcomings by controlling the apparatus at a fixed angle or limiting the available range of motion for the insertion tool (i.e., a needle) which significantly reduces risk and prevents common mistakes.

In an example, an apparatus of the present disclosure is made of a sterile and reusable material, such as plastic, metal, or other material including materials having antimicrobial properties like copper. The apparatus includes a plurality of ports or pathways to receive needles of different sizes or gauges. The apparatus is configured to allow a needle to be inserted at a predetermined desired angle and to maintain that angle throughout the medical procedure. The apparatus may further generate indications (e.g. clicking or resistance) to a user when the needle is injected to a target location.

The apparatus of the present disclosure can provide for smooth and reliable translation along three directions (i.e., length, width, and depth). The apparatus can attach directly to an imaging probe or the like, to a patient's skin or clothing, or both while also receiving an insertion tool like a needle. The apparatus can improve precision, stability, and efficiency of an existing biopsy procedure or the like. The improvement can be achieved by maintaining a precise angle, like a 45-degree angle stick to maintain throughout a procedure. Moreover, during a procedure, the needle can be locked within a biopsy needle apparatus (channel) at a plurality of positions to obtain a biopsy in different locations of tissue to identify various conditions of the target tissue. For example, for a breast biopsy, multiple samples at various depths and locations can be obtained to identify invasive breast malignancies, fibrocystic changes in the breast, fat necrosis, etc. This can result in a decrease in surgical adverse events by augmenting stability through use of an imaging tool like an ultrasonic probe, robust skin clamps, and variable needle depth settings.

In an example, a user can insert a needle into a single-holed entry side. The needle is then guided into a singular channel that could translate on an opposite side of the apparatus to result in various angles extending from the entry side. At the exit side, the needle is locked in place within the movable channel by one of the numerous brackets on the exit side. In an example, five brackets are provided, creating at least five angles of penetration for the needle. Those brackets on the exit side can be circumscribed by five holes, from which the needle would exit the apparatus and enter a target tissue/location of a patient. The adjustable movement of the channel prior to locking into the brackets can be controlled manually by freehanded motion or as through an adjustment feature like a semi-circular wheel on the device. The entry port on the entry side can further include an adjustment screw which can be manually adjusted to limit the space within the opening. The screw is configured to accommodate various needle sizes (gauges) by bracing the needle against an interior edge of the entry port, and further locking the needle into the channel.

In an alternative example, the apparatus includes a plurality of independent stationary or fixed channels, which are the same width and depth, that emerge from the singular hole on the entry-side. In this example, there could be five brackets circumscribed by five holes on the exit side.

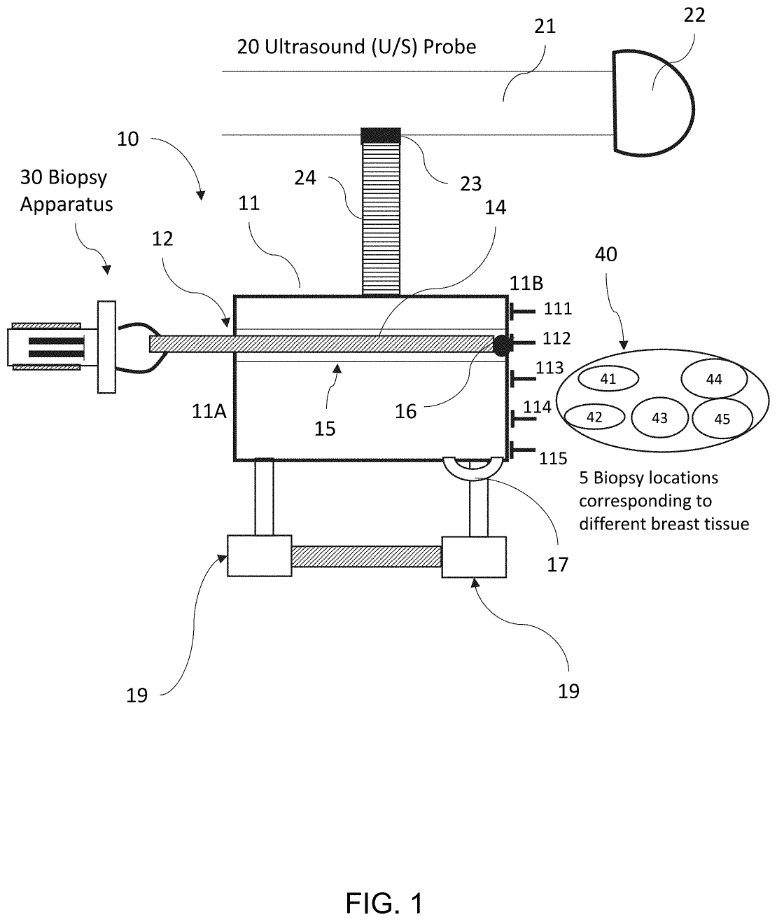

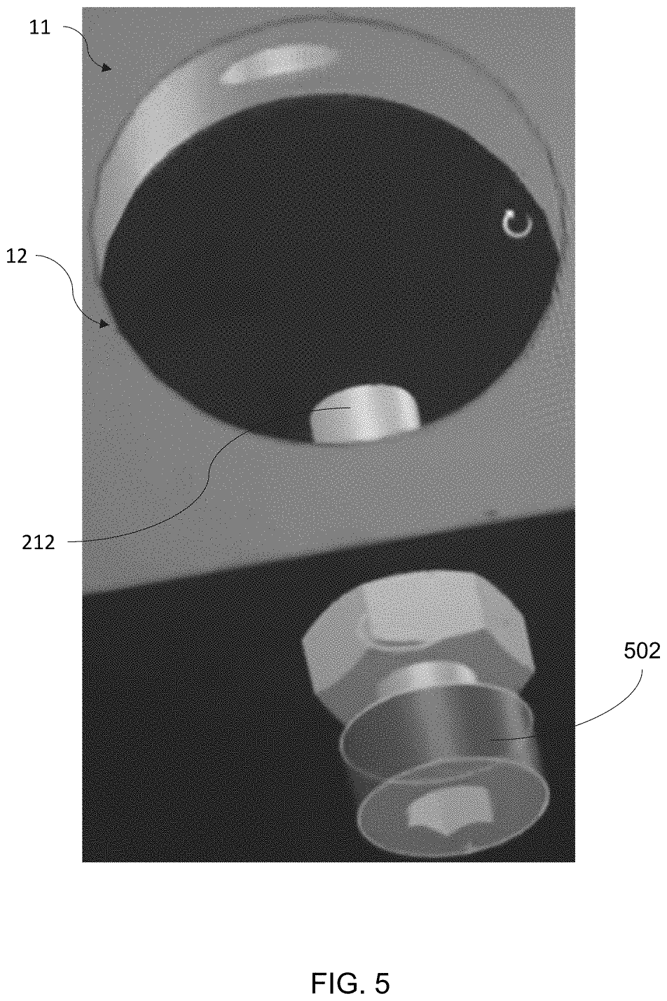

Referring to FIG. 1-5, the present disclosure provides for an insertion tool guide apparatus 10. FIGS. 1-3D illustrate an example apparatus 10 according to the present disclosure having an entry port 12 provided on an entry side 11A of an enclosure or casing 11. The internal portion of casing 11 can be made hollow to avoid any mechanical obstruction to the insertion device 14 as it passes from the entry port 12 to the exit position on the opposite end 11B. The entry port 12 can be configured with an adjustment tool 212 (See FIG. 5) to adjust for the gauge of insertion tool 14. Insertion tool 14 will interchangeably be referred to a needle 14 but it is within the scope of the disclosure to understand that any desired medical tool for insertion purposes can be used including but not limited to a cannula, a catheter, a needle, a biopsy needle, and others.

In this example, a plurality of receiving brackets 111-115 are provided at an exit side 11B. These brackets are configured to lock the insertion tool 14 in place at a desired angle and/or depth. For purposes of illustrating use, a biopsy device 30 can be provided to direct, guide and actuate the insertion tool 14. Device 30 can be designed to obtain tissue samples once reaching a target location 40. In this example, target location 40 is a breast tissue of a patient having five target locations 41-45.

Each biopsy location 41-45 can define its own position on the breast and/or a unique depth as compared to the other locations. Accordingly, the user can insert the insertion tool 14 through the entry port 12 and into the adjustable channel 15 and exit through any of the exit brackets or ports 111-115 to obtain five corresponding biopsy samples. In this example, the adjustable channel 15 can anchor at the entry port 12 and adjust at various angles towards the exit ports 111-115. This results in efficient, accurate, sampling with reduced error and reduced physical risk to the patient. Apparatus 10 can further include a mechanical dial 17 that engages an adjustment wheel 16 that can translate the exit end of the channel 15 to a desired bracket 111-115. The rolling of the dial can induce inferior/caudal movement and also control depth of insertion.

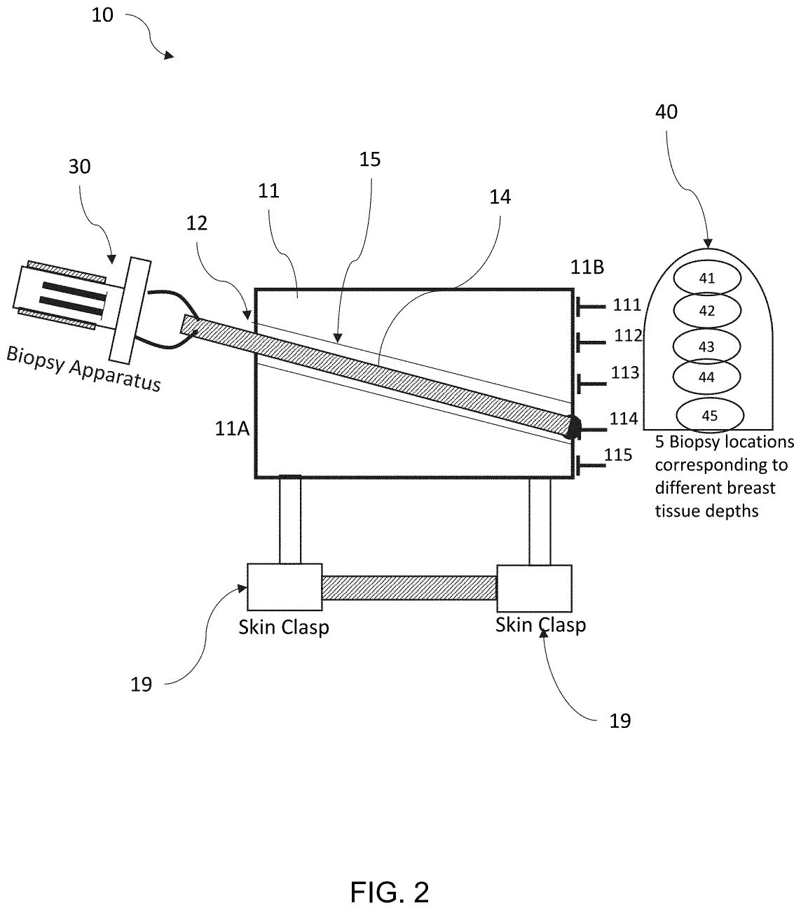

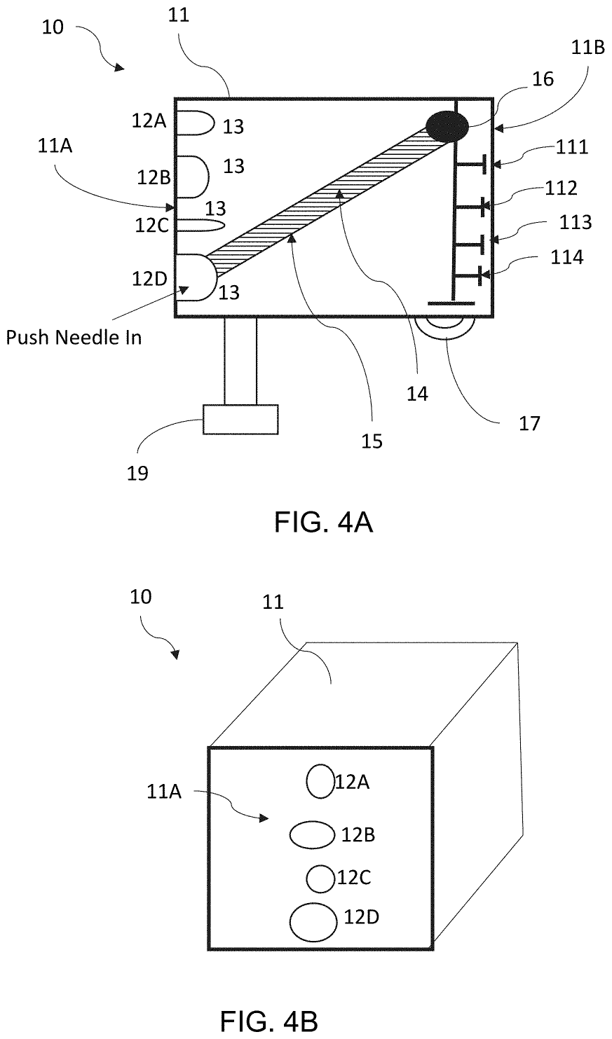

Apparatus 10 further includes at least one patient clasp device 19 that extends from a side of casing 11. In this example, clasp device 19 can be a sterile, plastic clasp capable of attaching to a patient's skin or adjacent to a target area for receiving the insertion tool 14. The device 19 is spaced off from casing 11 a sufficient distance and angle to ensure effective insertion tool 14 placement relevant to a target location. In an example, apparatus 10 can also include a single clasp 19 (FIG. 4A). In another example (FIGS. 1 and 2), a pair of spaced apart clasps 19 are provided for additional stabilization. Clasps 19 are configured to secure device 10 to a corresponding patient's skin or clothing to stabilize device 10 and thus provide for reliable guidance of the biopsy apparatus 30 and corresponding needle 12.

Apparatus 10 can be configured to securely mount or connect to an ultrasound probe 20. Probe 20 can include an elongated probe body 21 and transducer 22. In an example, apparatus 10 includes a connector clasp 23 connecting to the probe body 21. Probe body 21 can be any suitable construction to allow for manual maneuverability of the transducer 22 over a target surface to provide desired imaging. Probe body 21 can be an elongated shaft or handle that is relatively strong and is held or gripped by a user. Extending from the clasp 23 is an adjustable plastic piece 24 (mounting arm) configured to determine the length between the probe 20 and casing 11. This is configured to secure the casing 11 in place relative to a target 40 and is anchored to the imaging device, such as probe 20. It can also function to lock casing 11 in place relative to the probe 20.

In the example of FIG. 1, biopsy tool 30 is shown directing a needle 14 to access tissue 40 at fixed, predetermined angles to obtain tissue samples (41-45). In this example, the adjustable channel 15 extends from the entry port 12 to exit bracket 112 to obtain a sample 42. In FIG. 2, the adjustable channel 15 and the corresponding biopsy needle 14 extends from the entry port 12 to exit bracket 114 at a different angle as compared to FIG. 1 to obtain a sample 44. The target tissue 40 and corresponding target locations 41-45 can be the same or different from patient to patient or procedure to procedure. These examples are meant to illustrate variations of the apparatus 10 schematically. Variations in locations and depths can be made accordingly. Moreover, the adjustment tool 212 (FIG. 5) positioned in entry port 12 can restrict or allow for variation in needle 14 gauge and catheter/sheath sizes (i.e., thicknesses) through automatic or manual adjustment. In the example of FIG. 5, the adjustment tool is an adjustment screw 212 that can be manually adjusted by a user. The size or area of the opening defined by port 12 can be adjusted accordingly. In another example, the port adjustment with adjustment screw 212 is configured with an auditory indicator like a clicking sound to help identify when a size change has occurred. Moreover, a dial or other visual indicia can be provided to indicate gauge and depth settings.

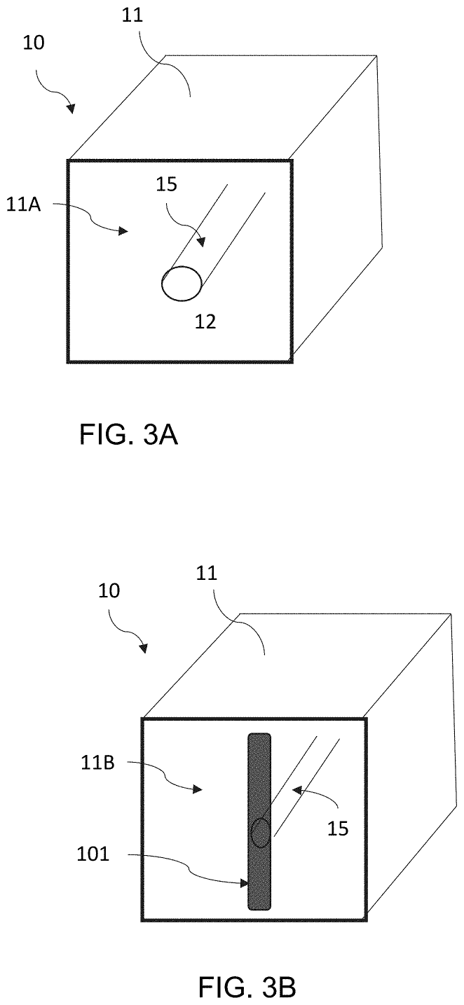

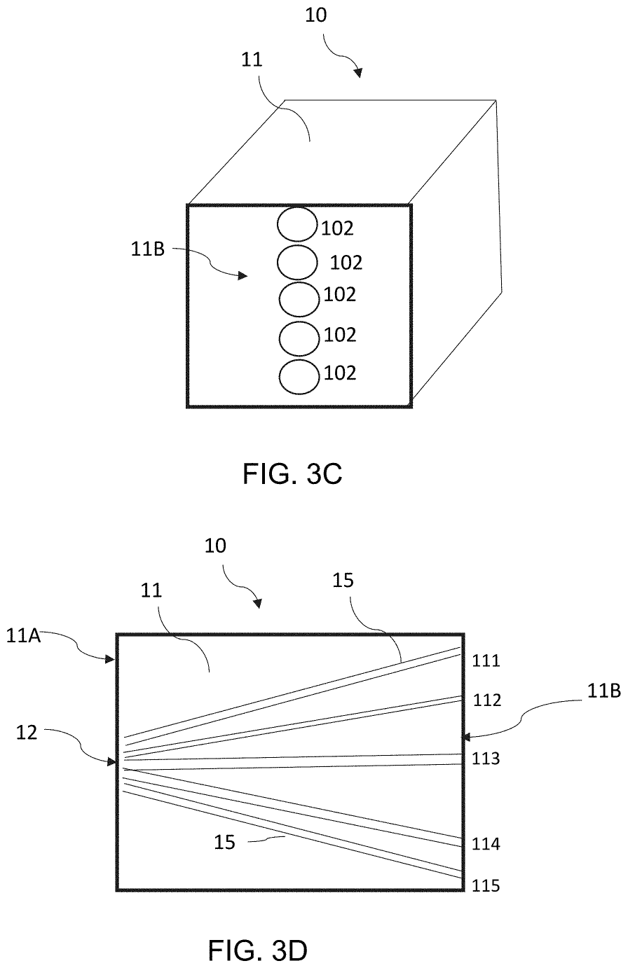

FIGS. 3A-3D illustrate schematic entry side 11A and exit side 11B variations of the guide apparatus 10. In this example, guide apparatus 10 includes a casing 11 for an enclosure having an entry port 12 provided on an entry side 11A. Adjustable channel 15 extends from entry port 12 to the opposite exit side 11B. FIG. 3B illustrates an exit side 11B defining an exit channel 101. The receiving brackets 111-115 are provided within exit channel 101 to allow the needle 14 to exit and access the target tissue. In the example of FIG. 3C, a plurality of fixed exit ports 102 are provided as an alternative to an exit channel 101. In yet another example of FIG. 3D, a plurality of fixed channels 15 are provided in apparatus 10 that extend from entry port 12 to opposite exit side 11B. Each fixed channel 15 engages a corresponding bracket 111-115 to allow passage through either an exit channel 101 (FIG. 3B) or a plurality of exit ports 102 (FIG. 3C). Engagement and disengagement of the needle 14 and channel 15 from the brackets 111-115 should be configured to be sturdy yet relatively easy for a user or medical professional.

It is further contemplated that a fully robotic and/or electrical mechanism is provided to automate the channel and angle adjustments and/or needle size (gauge) adjustment). In this example, a controller is provided to automate the process and adjust any mechanical adjustment mechanisms. It is further contemplated that apparatus 10 further includes at least one sensor and/or communication module to transmit signals and sensor data related to the functionality, operation life, and/or position information related to the channel, the needle or any corresponding parts. In an example, a user can be looking at a screen displaying an ultrasonic view. The sensors could interface with the screen and the device to coordinate positioning and provide real-time data like insertion depth, insertion angle, etc.

In FIGS. 4A-4B, another example of guide apparatus 10 is shown schematically in a cross-section or transparent view to show internal structural components. In FIG. 4A, apparatus 10 includes a casing 11 that defines a plurality of entry ports 12A, 12B, 12C, 12D are spaced apart from each other and configured to receive variations of needle sizes and thicknesses (gauges). In this example, casing 11 is an enclosure structure like an enclosed box having an entry side 11A for receiving an insertion tool 14 and an exit side 11B or guiding the insertion tool 14 to a target area, such as the subcutaneous tissue of a patient.

In this example, four entry ports 12A-12D are defined on entry side 11A. Each entry port 12A-12D can include a keyed guide piece 13 provided therein and spaced apart to determine the depth of a needle stick. Each piece 13 can be made of plastic and provide resistance to ensure desired insertion of the insertion tool 14. A clicking or audible mechanism can be provided in each guide piece 13 that is configured to be heard upon desired angle and depth of needle 14 through each port. An indicator mechanism can include zippers or brackets that would let a user know audibly or through tactile feedback how deep the needle is through clicks, vibration, and/or mild resistance. In one example, the entry ports 12A-12D are spaced apart about 2 mm from each other. In another example, each entry port 12A-12D defines a different diameter and thus corresponds like a key to the desired needle or insertion tool 14 of varying thicknesses. In this example, the needle 14 passes through an adjustable channel 15 which can be a catheter or a sheath that can adjust a fixed angle for directing and guiding needle 14. Channel 15 extends from at least one of the entry ports 12A-12D to an exit port 102 (See FIG. 3C) or channel 101 (See FIG. 3B) formed on the opposite exit side 11B. At the exit, a plurality of brackets 111-114 can be provided to lock the channel 15 in place and thus the needle 14 in a secure angle of penetration.

In an example, a casing is connected to or mounted to an ultrasound probe. The casing can be connected to a probe by any connection means sufficient to secure the apparatus 10 in a fixed position relative to the probe. This allows for simultaneous imaging of a target area and subsequent needle insertion and monitoring. A clamp is provided that extends from a different side of the casing. The clamp is configured to secure the apparatus to a patient via their skin or clothing.

Regarding FIG. 5, an adjustment tool 212 is provided to extend into entry port 12. The adjustment tool 212 can be manually twisted or adjusted to reduce the available entry space defined by entry port 12. This can secure an insertion tool 14 (like a needle or catheter) in place during use, but also adjust for varying needle sizes (gauges).

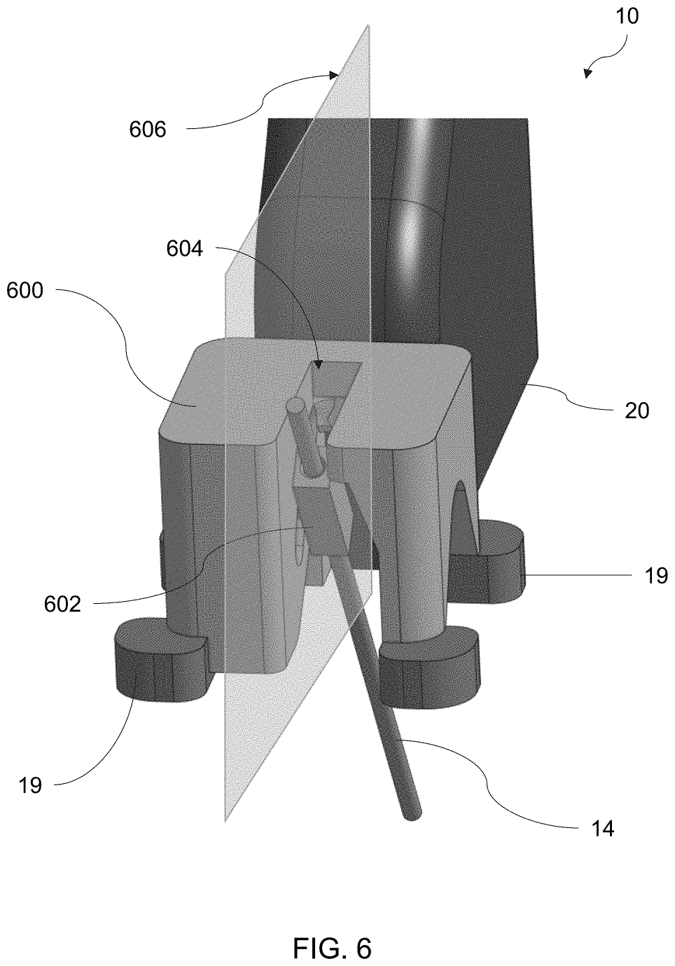

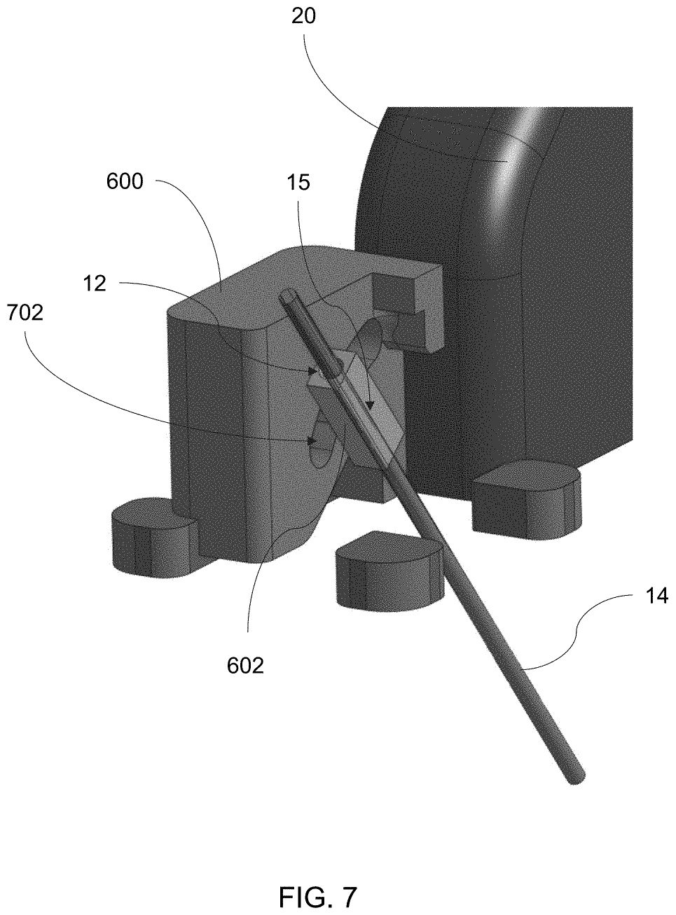

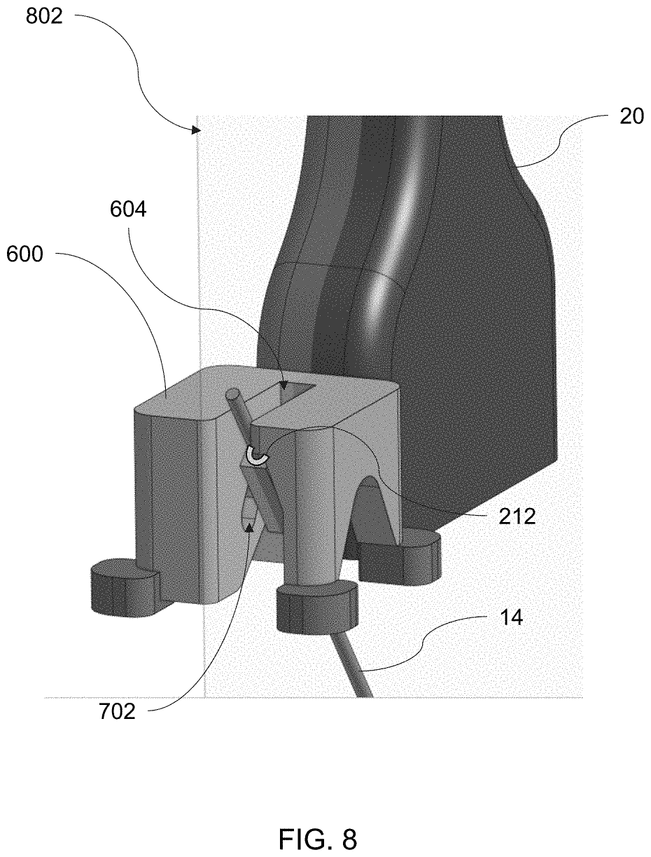

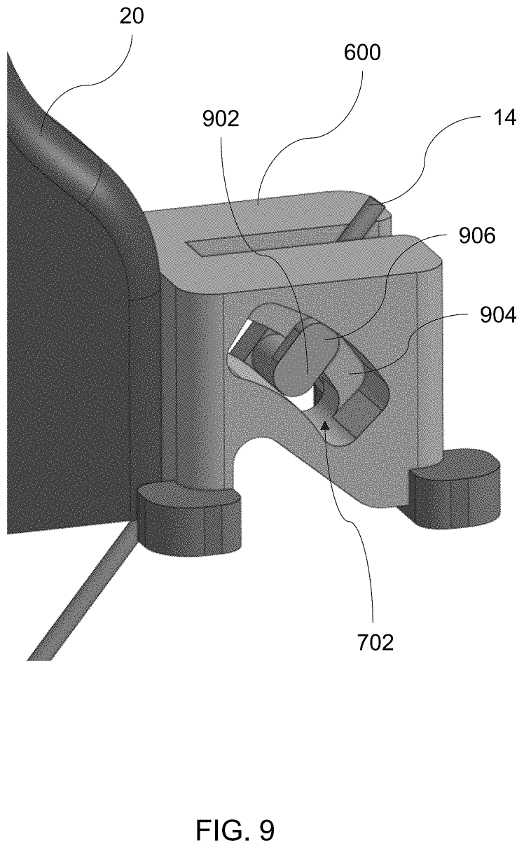

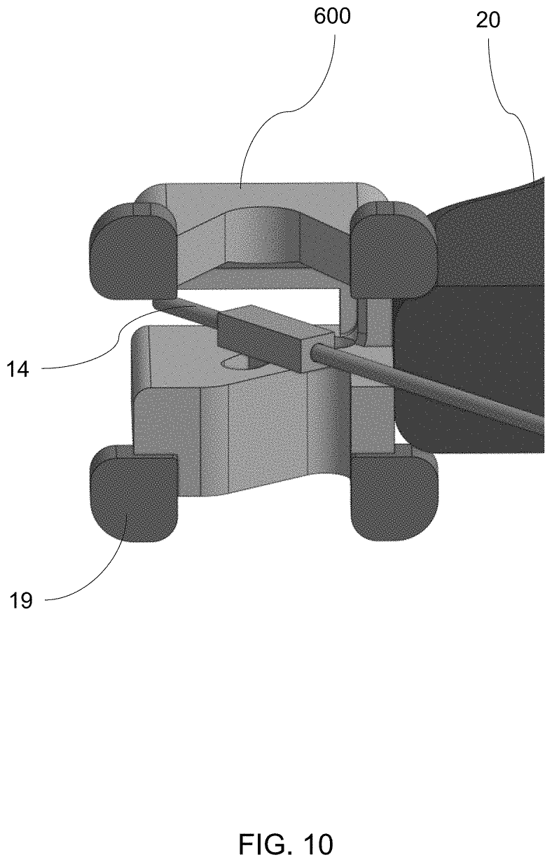

Referring to FIGS. 6-12, in an example, a frame 600 is provided defining a guide channel 702 along a side portion of the frame 600. The frame 600 can be manufactured or in another example, may be 3D printed with any sufficient material. In a further example, the frame can be made or 3D printed from a copper-infused material providing anti-microbial properties to the frame 600 while still safely allowing it to be in constant contact with a patient throughout a procedure. In one example, frame 600 is configured to accommodate access to any interior components, hence providing ample space for removal of guide body 602.

While in use, the frame 600 can be configured to directly abut ultrasonic probe 20 to accommodate coordinated use with either parallel or perpendicular ultrasound-guided techniques. Abutting probe 20 to the frame 600 confers increased stability to the probe 20 and to the frame 600. When probe 20 is stabilized, an ultrasonic plane of view 802 (schematically shown) is also stabilized. In one example, the frame 600 defines a main channel 604 configured to provide enough space for guide body 602 to rotate. The main channel 604 allows for the rotational plane of motion 606 for guide body 602 to be aligned with the ultrasonic plane of view 802.

In an example, frame 600 is configured to restrict the movement of a needle 14 such that when a needle is inserted into the apparatus 10 the needle remains confined within the plane of view 802 of the ultrasonic probe 20. The frame 600 can allow for needle 14 to be manipulated and translated in multiple directions but still maintain the needle within the ultrasonic plane of view 802. The plane of view 802 can then be interpreted to determine the position and depth of needle 14 and enable users to make concurrent micro-adjustments.

A plurality of feet 19 are positioned below frame 600 and secure apparatus 10 to the body of a patient. Feet 19 may be arranged on the corners of the frame 600 and can be made of a material that adheres to the skin of a patient, yet confers enough durability to last the duration of a procedure (e.g. biopsy). In an example, feet 19 are configured to receive adhesive gel or are granted additional stability by medical tape. Feet 19 can provide increased friction with the patient's skin to stabilize the apparatus 10, therefore decreasing the risk of injury by inadvertent slipping of apparatus 10 across the patient's skin. Feet 19 may also be sterilizable, removable, and interchangeable between procedures.

In an example, the frame 600 defines a guide channel 702 formed along one side of the frame 600. Channel 702 is configured to receive a movable guide body 602 and can define an arcuate geometry which enables radial movement along its length by a guide body 602. In one example, channel 702 includes a ridge 904 protruding out and away from either the upper or lower surface of the channel 702. The guide body 602 is mounted within channel 702 to allow for slidable movement along channel 702. Guide body 602 is secured within channel 702 by a peg 902 removably attached on an end of guide body 602 and extends through the channel 702. The peg 902 is configured such that an end opposite the guide body 602 includes a lip 906 parallel and adjacent to an exterior face of ridge 904. Lip 906 abuts the exterior face of ridge 904 which restricts lateral movement of the guide body 602 along the channel 702.

The peg 902 or guide body 602 may also include a bearing at a connection point. The bearing may be a ball bearing or the like and configured to allow for rotational movement of the guide body 602 about the longitudinal axis of the peg 902. The rotational movement provides an additional means of adjustment for a needle 14 during operation of the apparatus 10 along plane of movement 606. Movement is typically restricted to remain within the plane of vision 802 of an ultrasonic probe 20. Needle 14 may be adjusted by rotation or translation of the guide body 602 without the risk of inadvertently moving the needle 14 out of sight of the probe 20 during a procedure.

In an example, the guide body 602 is attached to the peg 902 which is configured to include a frame lock 1104. When the frame lock 1104 is engaged, needle 14, guide body 602, and peg 902 are locked to frame 600 such that translation or movement of needle 14 is fully restricted in all planes of motion. Once the frame lock 1104 is disengaged, the needle 14 may again be moved and translated along the original planes of motion.

Guide body 602 defines an adjustable channel 15. In one example, adjustable channel 15 is arranged within the guide body 602 extending from an entry port 12 along the insertion path and extending linearly down a length of the guide body 602. The guide body 602 may also define multiple adjustable channels 15 in parallel to house multiple needles 14 or alternative insertion paths. The adjustable channels 15 may also be of different sizes to accommodate needles of varying sizes and gauges.

In use, needle 14 is passed through entry port 12, through the length of the adjustable channel 15 and is inserted into a patient. The length of the needle 14 is sufficient to allow for a portion of the needle 14 to remain captured within the adjustable channel 15, yet still capable of being driven to a desired depth to access a target location within a patient.

The guide body 602 may be sized and shaped to receive a needle of any desired size. In one example, the apparatus 10 is used for Fine Needle Aspiration (“FNA”) in which the guide body 602 is configured to receive size 22-to-27-gauge needles. In another example, the apparatus 10 is used for Core Needle Biopsies in which the guide body 602 is configured to receive size 16-to-21 gauge needles.

The guide body 602 may also provide for an adjustment tool 212 arranged on entry port 12 and configured to adjust the radius of the entry port 12 to accommodate a variety of sizes of needles or insertion tools. In one example, the adjustment tool 212 may be a screw which upon rotation is configured to adjust the size of a bushing arranged in the entry port 12.

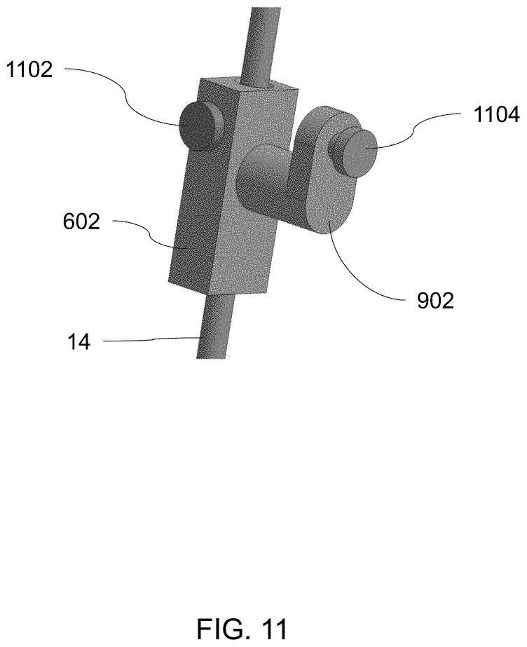

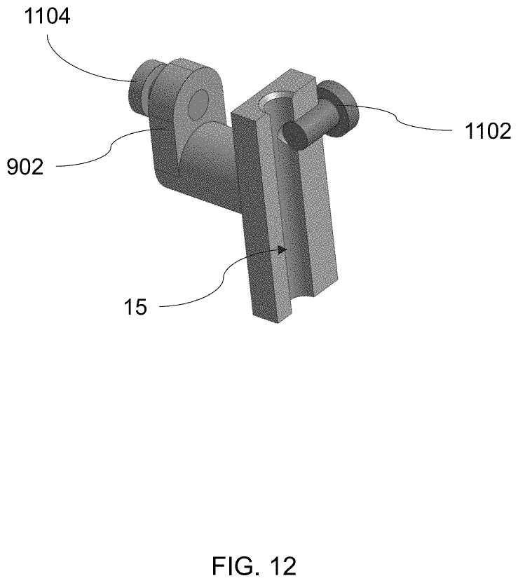

FIGS. 11 and 12 illustrates an example in which adjustable channel 15 provides for a lock 1102 on guide body 602. The lock 1102 may be a rubber cam lock or a screw and may be located in or adjacent to the adjustable channel 15. Lock 1102 is configured to secure needle 14 in place during procedures, thus enabling access for more precise tissue samples at a plurality of depths.

In an example, needle 14 is inserted into entry port 12 of adjustable channel 15, through the length of the guide body 602, and of adjustable channel 15. Needle 14 is then inserted into a patient while a portion of needle 14 remains within channel 15 as a guide to prevent lateral or vertical movement. Once needle 14 reaches a desired depth, lock 1102 can be activated to secure needle 14 in position and prevent proximal or distal movement of needle 14. In an example, a screw operates as a lock 1102 by screwing directly into needle 14 thus preventing movement.

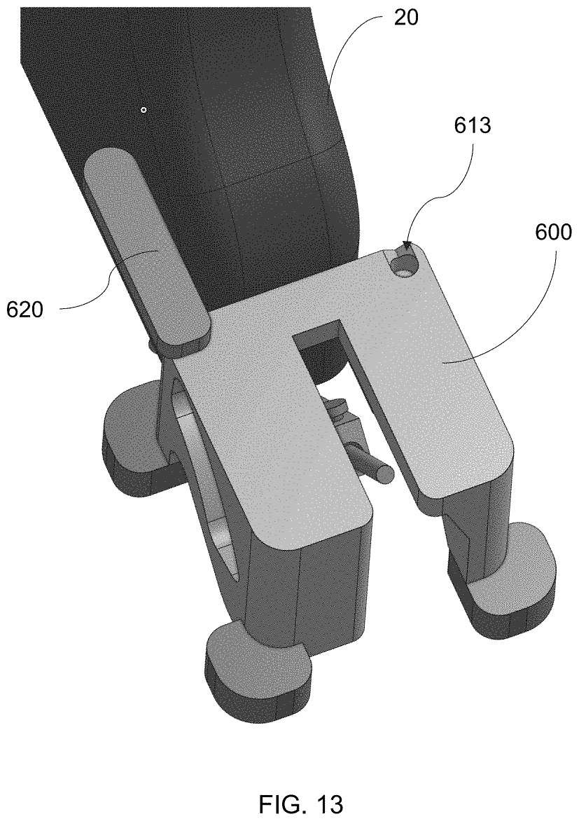

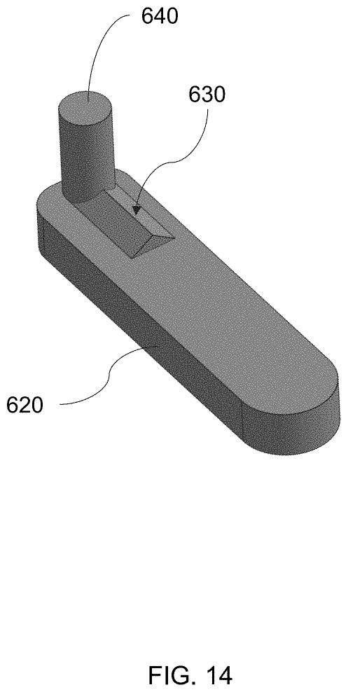

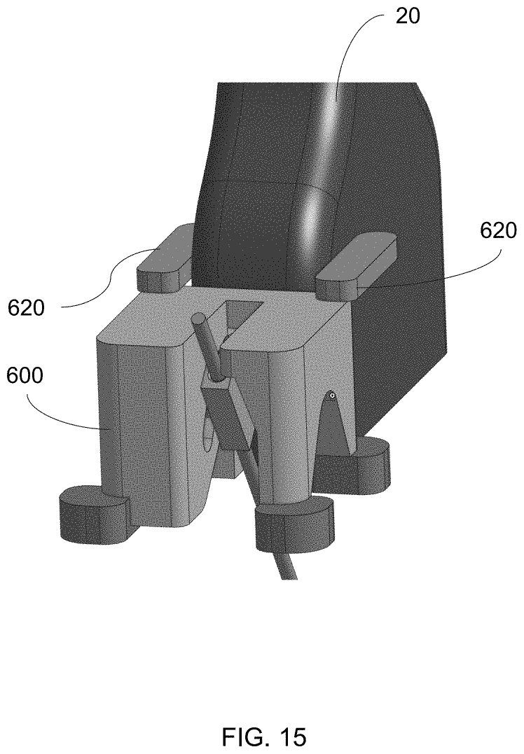

Referring to FIGS. 13-15, an example frame 600 is shown having an indent 613 or recess 613 formed on a corner frame 600. In this example, the indent 613 includes two ramped sections substantially making a V-shaped feature or “V-feature”. Indent 613 stabilizes arm 620 with an interfacing shape that are mating to align their respective objects. In an example, the mating geometries can be V-shape, circle, square, oval, triangle or hexagon. In an example, a receiving cavity extends down into frame 600 from the indent 613. The cavity is configured for receiving a connector peg 640 of an attachment arm 620. Extending from the peg 640 along a main body of arm 620 is a mating V-feature protrusion. Peg 640 extends in a perpendicular relationship to the main body of arm 620. When arm 620 is connected to frame 600, connector peg 640 is inserted into the receiving cavity of frame 600 and the V-feature indent 613 aligns and mates with protrusion 630.

The arms 620 can be freely inserted into the frame 600. In an example, the mating V-features 613 and 630 interlock. The “V” features 613 and 630 help align arms 620 and probe 20. However, in this example, the arms 620 do not fixate the probe 20 to frame 600. Arms 620 will be free to move after a certain force is applied.

In an example, an arm 620 or plurality of arms 620 attach to the frame 600. The arm(s) corral the probe 20 and ultrasonic plane of view 802 into alignment with the plane of movement 606. The arms 620 are attached in a manner which allows probe 20 to move independently of frame 600 after a certain amount of force is applied to the arms. This enables decoupling of the probe 20 from frame 600, in the instance the user moves probe 20 in an unintended manner. Without decoupling, frame 600 would move with probe 20 and in turn impart unintended movement on needle 14.

In an example medical procedure, such as an ultrasound-guided breast biopsy, an apparatus of the present disclosure can provide for: reducing complication rate, increasing diagnostic rate, curtailing quantity of local anesthetic delivery, and augmented micro-adjustability by the user. Lumps and/or abnormalities can sometimes be detected on physical examination, mammography, computerized tomography, or other imaging studies. However, medical professionals are not always able to identify if a mass is benign via imaging or examination. Hence, a breast biopsy is performed to remove tissue samples for examination under a microscope by a pathologist. An ultrasound uses a probe or transducer to generate and receive reflected sound waves to produce an image of a target region of the body. This can be a less invasive procedure in comparison to a non-image guided surgical biopsy. The device confers a heightened level of safety to the patient with reduction of tissue trauma and ensuant scarring. Moreover, the device can be reliably used to acquire tissue samples from which pathologists determine the presence of benign or malignant properties.

In another example, computerized tomography (CT)-guided coaxial biopsies (e.g. liver, lung, and kidney) necessitate the initial placement of a hollow guide needle which serves as a placeholder followed by a biopsy needle that, upon advancement, extracts the tissue sample. Device 10 enables the user to make micro adjustments to the guide needle, in real time, curtailing the amount of CT scans obtained to achieve accurate guide needle placement. This decreases cumulative radiation exposure to the patient in receipt of a CT-guided biopsy.

An example medical biopsy procedure can include, but is not limited to, the steps of: (1) numb a target region with local anesthesia; (2) create a small incision into the skin; (3) insert a biopsy needle and obtain a sample (repeat as needed to get multiple samples from various locations of the target region); (4) observe patient for a sufficient time period following procedure; and (5) then discharge.

One purpose of apparatus 10 is to maintain angle of entry throughout a procedure to increase precision, stability, and decrease complication rate. Width, length, and depth (X, Y, and Z) translatability can help ensure angle of entry as it may vary with any particular lesion. The translatability is helpful due to the variant locations of entry for the biopsy (i.e. Upper Outer Quadrant Biopsy vs Lower Inner Quadrant Biopsy), the depth and size of the lesion (to regulate depth and to avoid transecting anatomy crucial to life), as well as operator preference. Ultrasonic imaging as well as biopsy needle(s) both have built-in depth indicators, so the apparatus 10 can ensure safe and secure angulation for the breast biopsy procedure.

The foregoing description of various forms of the invention has been presented for purposes of illustration and description. It is not intended to be exhaustive or to limit the invention to the precise forms disclosed. Numerous modifications or variations are possible in light of the above teachings. The forms discussed were chosen and described to provide the best illustration of the principles of the invention and its practical application to thereby enable one of ordinary skill in the art to utilize the invention in various forms and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An insertion tool guide for a medical procedure comprising:
(a) a frame configured to be adjoined to a medical image modality and maintain an insertion angle aligned with a plane of view generated by the medical image modality;
(b) a guide body defining an entry port and an adjustable channel configured to receive and translate an insertion tool;
(c) a main channel defined by the frame configured to allow the guide body and the insertion tool to rotate within a plane of movement;
(d) a guide channel formed along one side of the frame configured to allow the guide body to translate along its length; and
(e) a peg extending from the guide body and through the guide channel to secure the guide body within the frame;
wherein, the guide body is configured to rotate about an axis defined by the peg.

2. The insertion tool guide of claim 1, wherein the insertion tool is selected from the group consisting of a needle, a biopsy needle, a cannula, a catheter, and combinations thereof.

3. The insertion tool guide of claim 1, wherein the medical image modality is selected from the group consisting of an ultrasound having an ultrasound probe, a computerized tomography (CT) scan, magnetic resonance imaging (MRI), a positron emission tomography (PET) scan, and combinations thereof.

4. The insertion tool guide of claim 1, wherein the medical image modality is an ultrasound having an ultrasound probe and the tool guide further includes one or more attachment arms extending from the frame to contact and guide the ultrasound probe to its intended position in relation to the frame.

5. The insertion tool guide of claim 4, wherein the attachment arm is configured to align the frame to maintain the insertion tool within the plane of view generated by the ultrasound probe.

6. The insertion tool guide of claim 5, wherein the attachment arm is configured to withstand a preset amount of force applied to the probe to maintain the frame in connection to the probe and disengage from the frame when the force surpasses the preset amount preventing unintended movement of the insertion tool during use.

7. The insertion tool guide of claim 5, wherein the attachment arm includes a connector peg and defines a protruding V-feature for matching a mating indent V-feature defined on the frame.

8. The insertion tool guide of claim 1, further comprising a lock extending from the peg configured to prevent translational movement of the insertion tool as it passes through the adjustable channel and configured to be activated and restrict or prevent movement of the insertion tool.

9. The insertion tool guide of claim 1 wherein, the guide body is configured to rotate about an axis defined by the peg.

10. The insertion tool guide of claim 1, further comprises a removable guide body that is interchangeable with a different guide body defining a different diameter of the entry port to accommodate a corresponding gauge of an insertion tool.

11. The insertion tool guide of claim 1, wherein the peg secures the guide body to the frame such that the guide body is configured to slidably travel through the guide channel.

12. The insertion tool guide of claim 1, wherein the peg is removably attached to the guide body and the guide body is removably attached to the frame.

13. The insertion tool guide of claim 1, further comprising a plurality of feet positioned on a lower side of the frame formed of a material configured to adhere to a surface of a target area, wherein the feet are optionally removable.

14. The insertion tool guide of claim 1, wherein the insertion tool is a biopsy needle configured for use with a biopsy procedure.

15. The insertion tool guide of claim 14, wherein the biopsy procedure is configured to obtain a biopsy sample of a target tissue selected from the group consisting of a liver, a breast, lung, kidney, thyroid, and combinations thereof.

16. A medical biopsy process using the insertion tool guide of claim 14, the process comprising:
(a) providing the insertion tool guide and the biopsy needle;
(b) making a small incision on a tissue surface to allow for biopsy needle to access a target region;
(c) inserting the biopsy needle using the insertion tool guide and a corresponding medical image modality; and
(d) obtaining a sample of the target region.

17. The process of claim 16, further comprising a repeating step of inserting the biopsy needle to obtain a plurality of samples from different locations of the target region.

18. The process of claim 16, wherein the biopsy procedure is configured to obtain a biopsy sample of a target tissue selected from the group consisting of a liver, a breast, lung, kidney, thyroid, and combinations thereof.

19. The process of claim 16, wherein the biopsy procedure is a coaxial biopsy including a step of inserting and placing a guide needle prior to threading a biopsy needle through the guide needle in order to obtain the sample.

20. An insertion tool guide system for a medical procedure comprising:
(a) a frame configured to be adjoined to a medical image modality and maintain an insertion angle aligned with a plane of view generated by the medical image modality;
(b) a guide body defining an entry port and an adjustable channel configured to receive and translate a biopsy needle in a first direction;
(c) a main channel defined by the frame configured to allow the biopsy needle to rotate within a plane of movement;
(d) a guide channel formed along one side of the frame configured to translate the guide body along its length;
(e) a peg extending through the guide channel and attached to the guide body, wherein the guide body is configured to rotate about an axis defined by the peg; and
(f) a lock configured to prevent translational movement of the biopsy needle as it progresses through the adjustable channel and to be activated and restrict or prevent movement of the insertion tool.
